(19) United States
(12) Patent Application Publication
(10) Pub. No.: US 2010/0087638 A1
(43) Pub. Date: Apr. 8, 2010

(54) PROCESS FOR THE PREPARATION OF LAMOTRIGINE

(76) Inventors: Jean-Paul Roduit, Grone (CH); Francis Djojo, Visp (CH)

Correspondence Address: HOFFMANN & BARON, LLP, 6900 JERICHO TURNPIKE, SYOSSET, NY, 11791, US

(21) Appl. No.: 12/374,936

(22) PCT Filed: Aug. 9, 2007
(86) PCT No.: PCT/EP07/07055

§ 371 Date: Nov. 19, 2009

(30) Foreign Application Priority Data
Aug. 14, 2006 (EP) 06016915
Aug. 31, 2006 (EP) 06018197
Nov. 17, 2006 (US) 60859514

(51) Int. Cl. C07D 253/06; C07C 253/00; C07C 255/00

(52) U.S. Cl. 544/182; 558/408

(57) ABSTRACT

A novel process for the preparation of lamotrigine and its intermediates is devised.

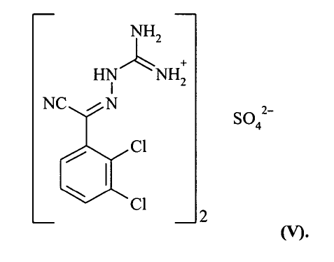

The present invention relates to a novel process for the preparation of lamotrigine and its intermediates.

Lamotrigine (3,5-diamino-6-(2,3-dichlorophenyl)-1,2,4-triazine) of formula (I) is a drug used for the treatment of disorders of the central nervous system (CNS), in particular for the treatment of epilepsy (cp. EP 0021121 A).

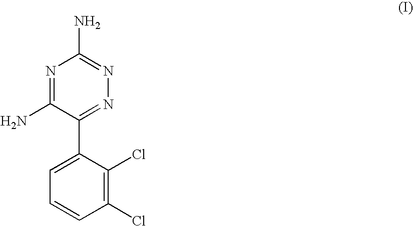

As lamotrigine has emerged to be one of the most successful anti-epileptic and anti-convulsant agents for treating CNS disorders, its commercial production has assumed greater significance. Whilst various processes of preparing lamotrigine are known in the art, there remains a need for a more efficient and environmentally friendly process, in particular related to waste production. Enhancing efficiency is also desirable with regard to yield as well as to reducing the overall processing time and the number of processing operations.

The prior art has devised a synthetic strategy which may be basically outlined as given below; in particular the intermediate condensation step proved critical with regard to yield and slow reaction rate (cp. WO 2004/039767):

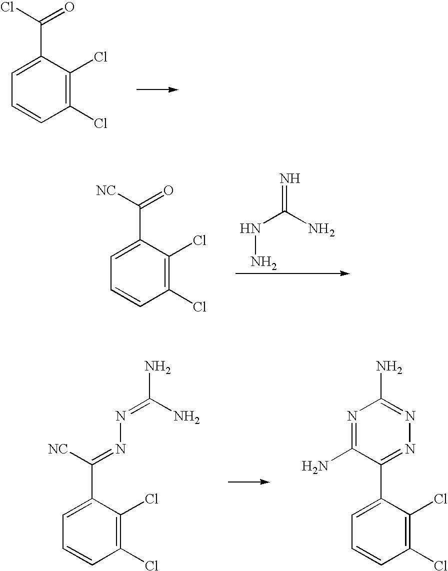

In the presence of water 2,3-dichlorobenzoyl cyanide is easily hydrolyzed to 2,3-dichloro-benzoic acid, which imposes restrictions on the solvent system and on the chemistry used in the condensation step with aminoguanidine as well as in its own synthesis. The processes described in WO 00/35888 and WO 01/49669 both use at least stoichiometric amounts of copper cyanide in organic solvent systems generating a large amount of copper-containing waste which is a major drawback for an industrial process from the perspective of waste treatment.

It is an object of the present invention to devise another, improved process for the synthesis of lamotrigine avoiding the disadvantages of the prior art. This object is achieved by the processes as laid down in the independent claims.

According to the present invention, it is devised a process of preparing a compound of formula

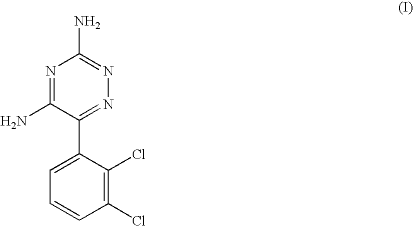

or a salt thereof, comprising the steps of:
(a) adding aminoguanidinium bicarbonate and a dehydrating agent selected from the group consisting of sulfur trioxide, oleum, disulfuric acid, a soluble disulfate salt, and phosphorus pentoxide, to a first polar solvent or solvent mixture,
(b) optionally removing at least part of said first polar solvent or solvent mixture,
(c) adding 2,3-dichlorobenzoyl cyanide of formula

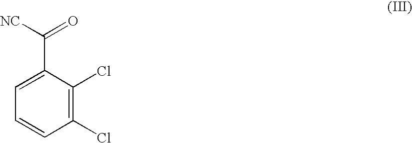

and reacting it in a second polar solvent or solvent mixture comprising an organic sulfonic acid selected from the group consisting of alkane-, arene-, arylalkane- or alkylarene-sulfonic acids, to yield a compound of formula

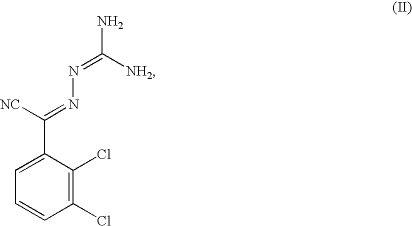

optionally in the form of its sulfate, phosphate, polyphosphate, tetrametaphosphate or hydrogensulfate salt, and
(d) cyclizing compound II in the presence of a base in a third polar organic solvent or solvent mixture to obtain compound I or a salt thereof.

In reaction step (a) preferably at least 0.5 equivalents of said dehydrating agent, more preferably from 1 to 1.5 equivalents of said dehydrating agent, are added per equivalent of aminoguanidinium bicarbonate.

In reaction step (d) compound II is preferably cyclized in the presence of an aqueous hydroxide, more preferably in the presence of an aqueous alkali metal hydroxide, most preferably in the presence of aqueous sodium hydroxide.

According to the present invention, it has surprisingly been found that adding sulfur trioxide (SO₃) or phosphorus pentoxide as a strong, irreversibly chemically dehydrating agent to aminoguanidinium bicarbonate prior to the addition of the second starting material of the condensation reaction (2,3-dichlorobenzoyl cyanide of formula III) in the continuing presence of preferably an excess of an anhydrous organic sulfonic acid such as methane-sulfonic acid is necessary and sufficient to enhance the yield and concurrently to strongly reduce the reaction time of the condensation.

According to the present invention the added sulfur trioxide is readily consumed in the dissolution process of the bicarbonate starting material, which first only dissolves slowly, drawn by the evolution of carbon dioxide. This way the present invention devises for the first time an efficient condensation process starting directly from aminoguanidinium bicarbonate undergoing a condensation reaction with 2,3-dichlorobenzoyl cyanide of formula III.

Whilst the use of essentially pure, liquid sulfur trioxide is strongly preferred according to the present invention, it is also possible to use oleum (sulfur trioxide dissolved in concentrated sulfuric acid) or disulfuric acid (H₂S₂O₇) as sources of sulfur trioxide. Disulfuric acid may optionally be used in the form of a metal disulfate salt being soluble in suitable first polar solvents according to the present invention such as, for example, sulfur dioxide (SO₂) or N,N-dimethylformamide (DMF).

Phosphorus pentoxide may also be used as a suitable dehydrating agent according to the present invention. The suitable dehydrating agents according to the present invention do not scavenge the dissolved aminoguanidine starting material even if used in slight excess of more than one equivalent per equivalent of aminoguanidinium bicarbonate.

Preferably, the first and second polar solvents are polar aprotic organic solvents or solvent mixtures or sulfur dioxide, more preferably water-miscible polar aprotic organic solvents or solvent mixtures or sulfur dioxide, most preferably selected from the group consisting of sulfolane (tetrahydrothiophen-1,1-dioxide), N-methylpyrrolidone, dimethylacetamide, dimethylformamide, tetrahydrofuran, dioxane, sulfur dioxide, dimethyl sulfoxide, and acetonitrile. Preferably at least 3, more preferably at least 7, most preferably at least 9 equivalents of the organic sulfonic acid or mixture of said organic sulfonic acids are present per equivalent of aminoguanidine starting material. The sulfonic acid or mixture of sulfonic acids is preferably essentially anhydrous. Preferably the first polar solvent or solvent mixture also comprises an organic sulfonic acid selected from the group consisting of alkane-, arene-, arylalkane- or alkylarenesulfonic acids. Examples are methanesulfonic acid, ethanesulfonic acid, propanesulfonic acid, p-toluenesulfonic acid, and benzene-sulfonic acid. More preferably the organic sulfonic acid is a C₁ to C₃ alkanesulfonic acid. Most preferably the organic sulfonic acid is methanesulfonic acid.

According to the present invention, the polar solvent also includes said organic sulfonic acid. The organic sulfonic acid may constitute the only solvent used in reaction steps (c) and/or (a). The presence of an organic sulfonic acid is essential for reaction step (c), the condensation reaction. For reaction step (a), the dissolution of the aminoguanidinium bicarbonate, the presence of an organic sulfonic acid is a preferred embodiment.

The dissolution of the aminoguanidinium bicarbonate may be performed in any polar solvent according to the present invention, preferably in acetonitrile or sulfur dioxide, more preferably in acetonitrile, mandatorily in the presence of a dehydrating agent. To avoid dilution effects upon addition of the organic sulfonic acid in reaction step (c), the solvent may be removed by standard evaporation techniques in an optional intermediate step (b).

Preferably the first polar solvent or solvent mixture also comprises an organic sulfonic acid as defined for step (c), more preferably it is the same organic sulfonic acid. Preferably the second polar solvent is said organic sulfonic acid itself, more preferably both the first and the second polar solvent is the same organic sulfonic acid, meaning that preferably at least in step (c), more preferably in both steps (a) and (c), the reaction mixture is free of any additional solvent. This embodiment, in which the organic sulfonic acid is the only solvent or reaction medium of steps (a) and (c) and the optional solvent removing step (b) can be omitted, is the most preferred embodiment of the process according to the present invention.

Preferably cyclization step (d) is carried out in a third polar aprotic organic solvent or solvent mixture, more preferably in the presence of acetonitrile, even more preferably in at least 50% (v/v) acetonitrile, most preferably in at least 80% (v/v) acetonitrile, preferably in the presence of an aqueous hydroxide, more preferably in the presence of an aqueous alkali metal hydroxide, most preferably in the presence of aqueous sodium hydroxide.

In a further preferred embodiment of reaction step (c), the intermediate of formula II is isolated by adding water to the reaction mixture and then precipitating the compound of formula II or its salt. By this method the compound of formula II can be obtained as a solid in the form of its salt precipitate, preferably in the form of its sulfate salt precipitate, by filtration or centrifugation. Said (substantially moist) salt precipitate can preferably directly be used as a starting material for cyclization step (d) without any additional drying.

The reaction temperature for the condensation step (c) is preferably in the range of from 25 to 60° C. Cyclization step (d) may be performed within a wide temperature range, preferably of from 5 to 200° C. The energy for the cyclization may be furnished either by heat or by irradiation (typically UV or microwave irradiation) or by a combination of these.

As a further improvement it is devised that 2,3-dichlorobenzoyl cyanide (formula III) can be prepared avoiding the use of large amounts of copper salts to render the complete route of synthesis more environmentally friendly. Catalysis by copper(I) is required to avoid an unwanted dimerization side reaction of the acid chloride at elevated temperatures. We have found unexpectedly that the cyanide-induced dimerization side reaction can be avoided to a great extent by adding only catalytic amounts of a copper(I) salt, preferably of copper(I) iodide, to the reaction mixture. Hydrogen cyanide or a cyanide salt is used as the cyanide source for the reaction, preferably an alkali metal or alkali earth metal cyanide, more preferably sodium cyanide.

According to the present invention 2,3-dichlorobenzoyl cyanide of formula III is furnished by reacting an acid chloride of formula

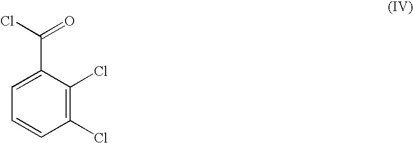

with a stoichiometric amount of hydrogen cyanide or a cyanide salt, with the proviso that said salt is not copper(I) cyanide or copper(II) cyanide, in the further presence of a catalytic amount of copper(I) iodide or of another copper(I) or copper(II) salt, with the proviso that, in case a copper salt other than copper(I) iodide is used, a second iodide salt is present in a catalytic or stoichiometric amount. Preferably said copper salt is present in an amount of 0.001 to 0.5 equivalents, more preferably in an amount of 0.01 to 0.1 equivalents, per equivalent of cyanide, which preferably is an alkali metal or alkali earth metal cyanide, more preferably sodium cyanide, which is used in at least a stoichiometric amount. More preferably said copper salt is copper(I) iodide or another copper(I) salt, most preferably it is copper(I) iodide. More preferably the reaction is carried out in a polar aprotic solvent or solvent mixture, most preferably in acetonitrile, under essentially water-free conditions.

The reaction rate and the extent of dimerization depend on the molar ratio of the used copper salt to the acid chloride. In case of copper(I) iodide, typically 4 to 5 mol-% are sufficient to achieve a convenient reaction rate at 20° C., while the rate of the dimerization side reaction can be kept at a very low level. The catalytic amount of copper(I) salt, preferably of copper(I) iodide, may either be added or be generated in situ using a suitable copper(II) salt in a reducing environment or suitable mixtures of copper(I) and copper(II) salts.

Traces of iodine are formed during the reaction and need to be removed before isolating the product in order to avoid an undesirable coloration. Iodine can be reduced to iodide using a variety of reagents, such as, for example, copper metal, sodium thiosulfate, sodium metabisulfite, sulfur dioxide. In the process of the present invention iodine is preferably reduced by sodium metabisulfite (Na₂S₂O₅).

2,3-Dichlorobenzoyl cyanide is a solid which can be crystallized from non-polar solvents such as hexane, heptane, or methylcyclohexane. However, the crystallization process has several drawbacks for a large-scale application: yield loss, need to recycle mother liquors, incomplete removal of the dimer impurity. We have found unexpectedly that 2,3-dichloro-benzoyl cyanide can be purified and isolated more efficiently by vacuum distillation. Typical distillation conditions are: pressure of from 2 to 20 mbar, boiling point of from 115 to 145° C.

The present invention comprises a further preferred embodiment of performing the condensation step (c) leading to the base N-guanyl-2-(2,3-dichlorophenyl)-2-imino-acetonitrile of formula II. Common salts (e.g. sulfate, mesylate, phosphate, nitrate) of compound II are hardly soluble in any solvent including water. Although they can be more easily separated by filtration than the free base, the isolation of the insoluble salts still requires handling a solid, which takes time and requires special precautions. The need to handle a solid intermediate is a drawback of all processes disclosed in the prior art. Therefore a further preferred embodiment of the present invention comprises the preparation and the use of salts of the base of formula II as well as of the aminoguanidine starting material that are readily soluble in polar organic solvents. A salt of the base of formula II which is easily dissolved in polar organic solvents results in a much better conversion rate of the cyclization reaction (d) and it also allows to perform the condensation step (c) and the cyclization step (d) in the same or a similar solvent system. Such a lipophilic salt can easily be isolated as a solid by addition of water and then to immediately be re-dissolved in the solvent system used for the cyclization reaction (d). Alternatively it is also possible to perform the condensation step (c) and the cyclization step (d) as a one-pot reaction without isolating the intermediate of formula II. Consequently, it is possible to perform the steps (a) to (d) as a one-pot reaction without isolating the intermediate of formula II when using the same solvent in the steps (a) and (c).

According to the present invention, it is also devised process of preparing a compound of formula

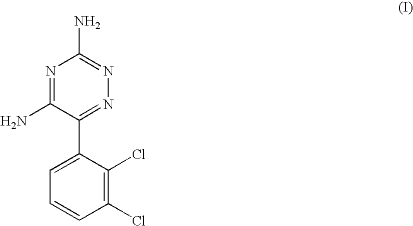

or a salt thereof, comprising the steps of:
(a) adding an aminoguanidinium tetrahaloborate or an aminoguanidinium tetraalkyl-, tetraaryl-, or tetra(alkylaryl)borate and further 2,3-dichlorobenzoyl cyanide of formula

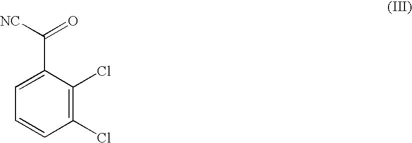

to a first polar organic solvent or solvent mixture and reacting it to yield a compound of formula

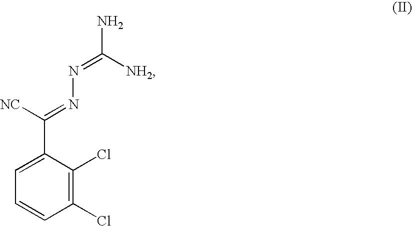

optionally in the form of its tetrahaloborate salt or its tetraalkyl-, tetraaryl-, or tetra(alkylaryl)borate salt after intermediate isolation, and
(b) cyclizing compound II in the presence of a base in a second polar organic solvent or solvent mixture to obtain compound I or a salt thereof.

Aminoguanidine is commercially available, for example, in the form of its bicarbonate salt. The bicarbonate has two important drawbacks for its use in the preparation process of lamotrigine according to the present invention. It is poorly soluble in both water and organic solvents, and it releases water and carbon dioxide from the decomposition of carbonic acid upon acidification (e.g. using tetrafluoroboric acid, scheme VI):

Aminoguanidine-H⁺.HCO₃⁻+2HBF₄→Aminoguanidine-H₂²⁺.(BF₄⁻)₂+CO₂+H₂O (VI).

Acidification of aminoguanidine with mineral acids usually results in a poorly soluble aminoguanidinium salts (e.g. sulfate, phosphate, etc.). This is surprisingly not the case with tetrafluoroboric acid (HBF₄), commonly also called fluoroboric acid, which is a stronger acid than hydrogen fluoride (HF). Aminoguanidinium di(tetrafluoroborate) is obtained from the bicarbonate as a hydrated salt which is easily soluble in polar organic solvents such as, for example, dimethyl sulfoxide (DMSO), dimethylformamide (DMF), dimethylacetamide, and preferably acetonitrile. For its preparation tetrafluoroboric acid can be used in the form of an aqueous solution or, preferably, in the form of an essentially anhydrous solution in an organic solvent. It is also possible to generate tetrafluoroboric acid in situ by dissolving an oxonium tetrafluoroborate, a solid that is easily soluble in most polar solvents.

Preferably, water is removed from the resulting reaction mixture by distillation. More preferably, water is distilled off as an azeotrope with a solvent having a lower boiling point than water. Most preferably, water is distilled off as an azeotrope with acetonitrile as described in example 7 of the present application.

In reaction step (b) compound II is preferably cyclized in the presence of an aqueous hydroxide, more preferably in the presence of an aqueous alkali metal hydroxide, most preferably in the presence of aqueous sodium hydroxide.

In a further preferred embodiment, the condensation step (a) and the cyclization step (b) are performed as a one-pot reaction without isolating the intermediate of formula II.

Lamotrigine obtainable according to any of the processes of the present invention can be further purified by crystallization from aqueous isopropanol and subsequent drying to obtain lamotrigine of pharmaceutical quality. It has been found a method of purifying lamotrigine by crystallization from a mixture of isopropanol and water, preferably from a mixture of isopropanol and water having a volume ratio of isopropanol:water of 3:1 to 2:1, more preferably having a volume ratio of about 2:1, yielding lamotrigine in an essentially anhydrous form. Lamotrigine is preferably obtained in an essentially anhydrous form having a water content of less than 0.1% (w/w), which can be determined, for example, by Karl-Fischer (KF) titration. Surprisingly this method has been found not to yield lamotrigine monohydrate in spite of the presence of water in the solvent mixture used for crystallization.

Further objects of the present invention are various stoichiometric salts of compound II that are obtained when precipitating the base from the reaction mixture by addition of water. Surprisingly it has been found that salt formation is highly selective, even if, for example, sulfate and sterically more demanding organic sulfonate anions may compete during the salt formation.

The salts can be of the stoichiometric composition L.X, wherein L is the singly protonated cation of compound II, and wherein X is a singly negatively charged anion of an acid selected from the group consisting of sulfuric acid, phosphoric acid, polyphosphoric acids, metaphosphoric acids, tetrafluoroboric acid, tetrachloroboric acid, tetraalkylboric acids, tetraarylboric acids, and tetra(alkylaryl)boric acids. Preferably X is a tetrafluoroborate or a tetraphenylborate ion.

The salts can also be of the stoichiometric composition L₂.X, wherein L is the singly protonated cation of compound II, and wherein X is a doubly negatively charged anion of an acid selected from the group consisting of sulfuric acid, phosphoric acid, polyphosphoric acids, and metaphosphoric acids. Preferably X is a sulfate ion.

It is evident to a person skilled in the art that the processes described in the present invention can be conveniently combined. A non-restrictive explanation of the present invention is provided by the following examples.

### EXAMPLES

### 1. Synthesis of 2,3-dichlorobenzoyl cyanide

2,3-Dichlorobenzoyl chloride (20.0 g, 100 mmol) and copper(I) iodide (0.90 g, 4.7 mmol) were suspended in acetonitrile (50 mL) and stirred at room temperature until a yellow homogeneous solution formed. Solid sodium cyanide (5.15 g, 110 mmol) was charged within 5 to 8 hours. After complete addition the reaction mixture was stirred for one hour, monitoring completion of the reaction by HPLC. The formed inorganic salts (mainly NaCl) were filtered off and washed with acetonitrile (15 mL). The acetonitrile was distilled off at reduced pressure (about 150 mbar). Sodium metabisulfite (Na₂S₂O₅, 0.4 g, 3 mmol) was added to remove traces of iodine. The product was finally isolated by vacuum distillation at 140° C. (jacket temperature), b.p. 115° C. (2 mbar).

The isolated yield of 2,3-dichlorobenzoyl cyanide (m.p. 60° C.) was ≧80% with a purity of 100% (according to analytical HPLC).

### 2. Synthesis of lamotrigine (3,5-diamino-6-(2,3-dichlorophenyl-1,2,4-triazine) via the sulfate salt

Aminoguanidinium bicarbonate (32.0 g, 235 mmol) was dissolved in methanesulfonic acid (85 mL) (some formation of carbon dioxide). Liquid sulfur trioxide (28.2 g, 352 mmol) was added dropwise at 20° C. during a period of about 20 minutes (vigorous evolution of carbon dioxide). Once emanation of gas had ceased, 2,3-dichlorobenzoyl cyanide (23.5 g, 117 mmol) was added and the reaction mixture was heated to 45° C. for 4 hours (in-process control: quantitative conversion, <1% of 2,3-dichlorobenzoyl cyanide). The reaction mixture was slowly poured into ice water (350 mL) yielding a white suspension which was cooled down to 10° C. and filtrated. The filter cake was washed with water (40 mL) which was subsequently removed to a large extent by suction of air through the filter. Without any additional drying the filter cake was directly used in the subsequent reaction step: it was suspended in a mixture of acetonitrile (190 mL) and water (60 mL), which had been pre-warmed to 50° C. An aqueous 25% (w/v) sodium hydroxide solution was added until a pH>12 was reached. The reaction mixture was heated to 70° C. for one hour whilst maintaining the pH. A clear, homogeneous solution formed. Afterwards the acetonitrile was removed quantitatively by vacuum distillation at 300 to 60 mbar and 45 to 80° C.

The resulting white suspension was cooled to 20° C. and filtrated. The filter cake was washed with water (2×15 mL) and dried under suction. Lamotrigine monohydrate (23.8 g, 87 mmol, 75%) was obtained after drying to constant weight at 60° C. in vacuo. Purity: 99.8% (analytical HPLC).

Lamotrigine of pharmaceutical quality, which is anhydrous, is obtained by recrystallization of crude lamotrigine from aqueous isopropanol and subsequent drying as laid down in example 4 of the present application.

### 3. Synthesis of Lamotrigine Via the Sulfate Salt as a One-Pot Reaction

Methanesulfonic acid (18.5 kg, 192 mol) was slowly added to a sulfur trioxide solution, 40% in methanesulfonic acid (10.5 L, 16.8 kg, 84.0 mol), at 25° C. Aminoguanidinium bicarbonate (8.57 kg, 63.0 mol) was charged in portions with stirring (vigorous evolution of carbon dioxide). The reaction was maintained at 25° C. for one hour, then 2,3-dichloro-benzoyl cyanide (8.40 kg, 42.0 mol) was added in portions. The reaction mixture was heated to 45° C. for 5 hours (in-process control: <1% of 2,3-dichlorobenzoyl cyanide) and subsequently cooled down to 30° C. Acetonitrile (68 L) was added and the yellow solution was slowly poured into an aqueous 25% (w/v) sodium hydroxide solution (65 L) at 30° C. (pH control: >12). After heating the reaction mixture to 70° C. for 3.5 hours the acetonitrile was removed by distillation at 200 mbar and 30 to 60° C., yielding an orange suspension which was allowed to cool down to 20° C. during one hour and maintained at this temperature for 30 minutes. The precipitated solid was separated by centrifugation, washed with water (2×19 L), and subsequently dried by further centrifugation, to obtain crude lamotrigine (9.1 kg).

### 4. Preparation of Anhydrous Lamotrigine

Crude lamotrigine (9.1 kg), suspended in a mixture of isopropanol (57 L) and water (18 L), was heated to 80° C. with stirring until a clear solution formed. The solution was filtered over activated charcoal on a heated filter. Water (9 L) was added and then the solution was cooled to 10° C. After 30 minutes the precipitated solid was separated by centrifugation, washed with a mixture of water (5 L) and isopropanol (11 L) at 10° C., and subsequently dried by further centrifugation. Then the product was dried in a dryer at 100° C. to obtain anhydrous lamotrigine (8.50 kg, 33.2 mol, <0.1% (w/w) of water according to KF titration).

### 5. Synthesis of Lamotrigine Via the Tetrafluoroborate Salt

A solution of aminoguanidinium tetrafluoroborate was freshly prepared from aminoguanidinium bicarbonate (2.42 g, 17.8 mmol) and anhydrous tetrafluoroboric acid, 53% (v/v) in diethylether (6.18 g), and diluted with acetonitrile (8 mL). 2,3-dichlorobenzoyl cyanide (1.50 g, 7.50 mmol) was added and the reaction mixture was heated to 45° C. for 4 hours.

In analogy to example 2 the reaction mixture was poured into ice water, yielding the tetrafluoroborate salt of compound II as a suspension which was cooled down to 10° C. and filtrated. The filter cake was directly dissolved from the filter at room temperature using essentially pure acetonitrile without any additional solvent. The subsequent cyclization step was performed as described in example 2.

### 6. Synthesis of Lamotrigine Via the Tetrafluoroborate Salt as a One-Pot Reaction

The condensation step was performed as described in example 5, with the exception that the isolation of the tetrafluoroborate intermediate was omitted. After the condensation step the solvents were removed on a rotary evaporator, then an equal volume of acetonitrile was added and the subsequent cyclization step was performed as described in example 2.

### 7. Synthesis of Lamotrigine Via the Tetrafluoroborate Salt with Azeotropic Removal of Water

To aminoguanidinium bicarbonate (30.0 g, 220 mmol), suspended in acetonitrile (400 mL), a solution of tetrafluoroboric acid, 50% (v/v) in water (78.75 g) was added dropwise at 15 to 30° C. within 10 to 30 minutes (strong evolution of carbon dioxide). A colorless solution formed. At ambient pressure and 77 to 83° C. acetonitrile (about 250 g) was removed by distillation (=ACN distillate 1) on a rotary evaporator. New acetonitrile (200 mL) was added to the residue, and according to the same procedure acetonitrile (about 160 g) was removed again (=ACN distillate 2), applying a slight vacuum at the very end to avoid an increase in temperature. The remaining solution (about 110 to 120 mL) was allowed to cool to 45° C. (in-process control: water content of <7%). A solution of 2,3-dichloro-benzoyl cyanide (22.0 g, 110 mmol) in acetonitrile (40 mL) was added and the reaction mixture was stirred at 45° C. for 5 to 6 hours (in-process control: <1% of 2,3-dichloro-benzoyl cyanide). Water (200 mL) was added to the white suspension and the acetonitrile was completely removed by vacuum distillation at 180 to 60 mbar and 35 to 45° C. The remaining viscous suspension was cooled down to 20° C. and filtrated. The filter cake was washed with water (20 to 40 mL), dried well under suction, transferred to a reaction vessel, and dissolved in ACN distillate 1 and/or 2 (80 to 150 mL). After heating to 65 to 70° C. an aqueous 7.5% (w/v) sodium hydroxide solution (106 mL) was added (pH control: >12.5). The reaction mixture was heated to 70 to 75° C. for one hour, then acetonitrile was removed by vacuum distillation at 300 to 60 mbar and 45 to 75° C. The resulting white suspension was cooled down to 18° C. and filtrated, the filter cake was washed with water (2×20 mL) and dried well under suction. Lamotrigine monohydrate (13.4 g, 49 mmol, 45%) was obtained after drying at 60° C. in vacuo. Purity: 99.8% (analytical HPLC).

### 8. Determination of Composition and Stoichiometry of the Isolated Salts of Compound II

The salts of N-guanyl-2-(2,3-dichlorophenyl)-2-imino-acetonitrile of formula II obtained as a solid from the filter cake in example 2 and example 7 were analyzed by ¹H-NMR to obtain a proof of structure.

### The Tetrafluorborate Salt of Example 7:

¹H-NMR (DMSO-d6): 7.55 (1H, m), 7.80 (1H, m), 7.88 (1H, m), 7.96 (5H, broad).

### The Sulfate Salt of Example 2:

¹H-NMR (DMSO-d6): 7.46 (1H, m), 7.70 (1H, m), 7.74 (1H, m), 7.17 (5H, broad).

¹H-NMR shows that this salt is not the methanesulfonate salt of compound II, which could theoretically also have been possible since the reaction was performed in methanesulfonic acid as a solvent.

To distinguish the sulfate from the hydrogensulfate salt and to determine stoichiometry, the proportion of sulfate was determined by standard ion chromatography (conductometric detection after hollow fibercounterflow borne suppression of eluent background). The amount of the anion was determined to be 12.79%, compared to the calculated amounts of 27.12% for [II-H⁺].[HSO₄⁻] and 15.74% for [II-H⁺]₂.[SO₄²]. Since the experimentally determined amount of the anion is very close to the calculated amount of the sulfate salt, it can be concluded that the intermediate of example 2 consists essentially of the sulfate salt of compound II (formula V):

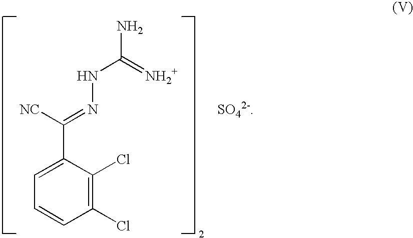

1. A process of preparing a compound of formula 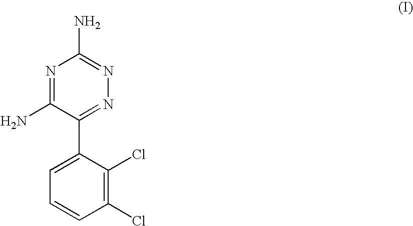
or a salt thereof, comprising the steps of:
(a) adding aminoguanidinium bicarbonate and a dehydrating agent selected from the group consisting of sulfur trioxide, oleum, disulfuric acid, a soluble disulfate salt, and phosphorus pentoxide, to a first polar solvent or solvent mixture,
(b) optionally removing at least part of said first polar solvent or solvent mixture,
(c) adding 2,3-dichlorobenzoyl cyanide of formula 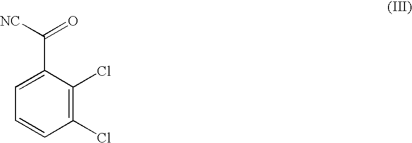
and reacting it in a second polar solvent or solvent mixture comprising an organic sulfonic acid selected from the group consisting of alkane-, arene-, arylalkane- or alkylarenesulfonic acids, to yield a compound of formula 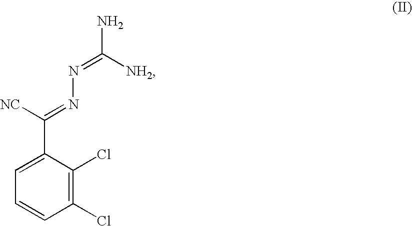
optionally in the form of its sulfate, phosphate, polyphosphate, tetrametaphosphate or hydrogensulfate salt, and
(d) cyclizing compound II in the presence of a base in a third polar organic solvent or solvent mixture to obtain compound I or a salt thereof.

2. The process of claim 1, wherein steps (a) to (d) are performed as a one-pot reaction without isolating the intermediate of formula II.

3. A process of preparing a compound of formula 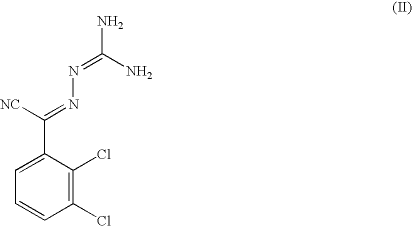
or a salt thereof, comprising the steps of:
(a) adding aminoguanidinium bicarbonate and a dehydrating agent selected from the group consisting of sulfur trioxide, oleum, disulfuric acid, a soluble disulfate salt, and phosphorus pentoxide, to a first polar solvent or solvent mixture,
(b) optionally removing at least a part of said first polar solvent or solvent mixture,
(c) adding 2,3-dichlorobenzoyl cyanide of formula 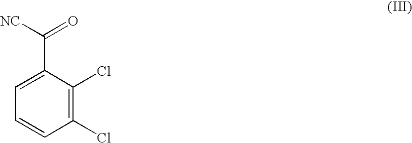
and reacting it in a second polar solvent or solvent mixture comprising an organic sulfonic acid selected from the group consisting of alkane-, arene-, arylalkane- or alkylarenesulfonic acids, to yield a compound of formula II, optionally in the form of its sulfate, phosphate, polyphosphate, tetrametaphosphate or hydrogensulfate salt.

4. The process of claim 1, wherein in step (a) an amount of at least 0.5 equivalents of said dehydrating agent per equivalent of aminoguanidinium bicarbonate is added.

5. The process of claim 1, wherein in step (a) an amount of 1 to 1.5 equivalents of said dehydrating agent per equivalent of aminoguanidinium bicarbonate is added.

6. The process of claim 1, wherein in step (d) compound II is cyclized in the presence of an aqueous hydroxide.

7. The process of claim 1, wherein in step (d) compound II is cyclized in the presence of an aqueous alkali metal hydroxide.

8. The process of claim 1, wherein in step (d) compound II is cyclized in the presence of aqueous sodium hydroxide.

9. The process of claim 1, wherein step (d) is performed in the presence of acetonitrile.

10. The process of claim 1, wherein step (d) is performed in at least 50% (v/v) acetonitrile.

11. The process of claim 1, wherein step (d) is performed in at least 80% (v/v) acetonitrile.

12. The process of claim 1, wherein compound I is isolated by adding water to the reaction mixture and then precipitating compound I or its salt as a solid.

13. The process of claim 1, wherein the first and/or second polar solvent or solvent mixture is a polar aprotic organic solvent.

14. The process of claim 1, wherein the first and/or second polar solvent or solvent mixture is a water-miscible polar aprotic organic solvent.

15. The process of claim 1, wherein the first and/or second polar solvent or solvent mixture is selected from the group consisting of sulfolane, N-methylpyrrolidone, dimethylacetamide, dimethylformamide, tetrahydrofuran, dioxane, sulfur dioxide, dimethyl sulfoxide, and acetonitrile.

16. The process of claim 1, wherein the first polar solvent or solvent mixture also comprises an organic sulfonic acid or a mixture of organic sulfonic acids.

17. The process of claim 1, wherein the first and/or second polar solvent or solvent mixture comprises at least 3 equivalents of an organic sulfonic acid or a mixture of organic sulfonic acids per equivalent of aminoguanidine starting material.

18. The process of claim 1, wherein the first and/or second polar solvent or solvent mixture comprises at least 7 equivalents of an organic sulfonic acid or a mixture of organic sulfonic acids per equivalent of aminoguanidine starting material.

19. The process of claim 1, wherein the first and/or second polar solvent or solvent mixture comprises at least 9 equivalents of an organic sulfonic acid or a mixture of organic sulfonic acids per equivalent of aminoguanidine starting material.

20. The process of claim 1, wherein the second polar solvent is an organic sulfonic acid.

21. The process of claim 1, wherein both the first and the second polar solvents are an organic sulfonic acid.

22. The process of claim 1, wherein both the first and the second polar solvents are the same organic sulfonic acid.

23. The process of claim 20, wherein the reaction mixture of condensation step (c) is free of any additional solvent.

24. The process of claim 20, wherein the reaction mixtures of both reaction steps (a) and (c) are free of any additional solvent.

25. The process of claim 1, wherein the organic sulfonic acid is essentially anhydrous.

26. The process of claim 1, wherein the organic sulfonic acid is an alkanesulfonic acid.

27. The process of claim 1, wherein the organic sulfonic acid is a Ci to C₃ alkanesulfonic acid.

28. The process of claim 1, wherein the organic sulfonic acid is methanesulfonic acid.

29. The process of claim 1, wherein compound II is isolated by adding water to the reaction mixture and then precipitating a salt of compound II as a solid.

30. The process of claim 1, wherein compound II is isolated by adding water to the reaction mixture and then precipitating a sulfate salt of compound II as a solid.

31. The process of claim 29, wherein the precipitated salt of compound II is separated by filtration or centrifugation.

32. The process of claim 29, wherein said isolated salt of compound II is directly used as a starting material for reaction step (d) without any additional drying.

33. The process of claim 1, wherein 2,3-dichlorobenzoyl cyanide of formula III is furnished by reacting an acid chloride of formula 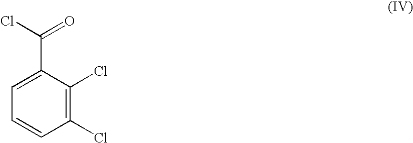
with a stoichiometric amount of hydrogen cyanide or a cyanide salt, with the proviso that said salt is not copper(I) cyanide or copper(II) cyanide, in the further presence of a catalytic amount of copper(I) iodide or of another copper(I) or copper(II) salt, with the proviso that, in case a copper salt other than copper(I) iodide is used, a second iodide salt is present in a catalytic or stoichiometric amount.

34. The process of claim 33, wherein said copper salt is present in an amount of 0.001 to 0.5 equivalents per equivalent of cyanide.

35. The process of claim 33, wherein said copper salt is present in an amount of 0.01 to 0.1 equivalents per equivalent of cyanide.

36. The process of claim 33, wherein said copper salt is copper(I) iodide or another copper(I) salt.

37. The process of claim 33, wherein said copper salt is copper(I) iodide.

38. The process of claim 33, wherein the reaction is carried out under essentially water-free conditions.

39. The process of claim 33, wherein the reaction is carried out in a polar aprotic solvent or solvent mixture.

40. The process of claim 33, wherein the reaction is carried out in acetonitrile.

41. The process of claim 33, wherein 2,3-dichlorobenzoyl cyanide is purified and isolated by vacuum distillation.

42. The process of claim 33, wherein 2,3-dichlorobenzoyl cyanide is purified and isolated by vacuum distillation at a pressure of from 2 to 20 mbar.

43. A process of preparing a compound of formula 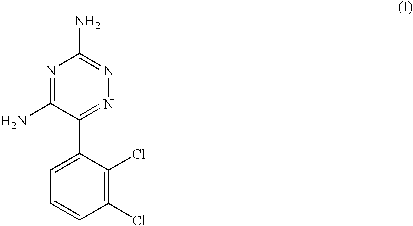
or a salt thereof, comprising the steps of:
(a) adding an aminoguanidinium tetrahaloborate or an aminoguanidinium tetraalkyl-, tetraaryl-, or tetra(alkylaryl)borate and further 2,3-dichlorobenzoyl cyanide of formula 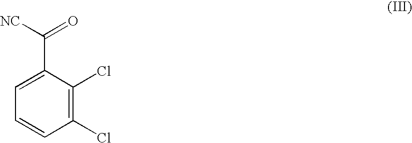
to a first polar organic solvent or solvent mixture and reacting it to yield a compound of formula 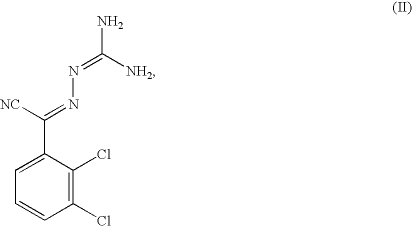
optionally in the form of its tetrahaloborate salt or its tetraalkyl-, tetraaryl-, or tetra(alkylaryl)borate salt after intermediate isolation, and
(b) cyclizing compound II in the presence of a base in a second polar organic solvent or solvent mixture to obtain compound I or a salt thereof.

44. The process of claim 43, wherein steps (a) and (b) are performed as a one-pot reaction without isolating the intermediate of formula II.

45. The process of claim 43, wherein the first polar organic solvent or solvent mixture is essentially anhydrous.

46. The process of claim 43, wherein the first and/or second polar organic solvent or solvent mixture is a water-miscible polar aprotic organic solvent.

47. The process of claim 43, wherein the first and/or second polar organic solvent is selected from the group consisting of acetonitrile, dimethylformamide, and dimethylacetamide.

48. The process of claim 43, wherein the first and/or second polar organic solvent is acetonitrile.

49. The process of claim 43, wherein step (b) is performed in the presence of an aqueous hydroxide.

50. The process of claim 43, wherein step (b) is performed in the presence of an aqueous alkali metal hydroxide.

51. The process of claim 43, wherein step (b) is performed in the presence of aqueous sodium hydroxide.

52. The process of claim 43, wherein the second polar organic solvent or solvent mixture is the same as the first polar organic solvent or solvent mixture.

53. The process of claim 43, wherein the aminoguanidinium tetrahaloborate is aminoguanidinium tetrafluoroborate or aminoguanidinium tetrachloroborate.

54. The process of claim 43, wherein the aminoguanidinium tetrahaloborate is aminoguanidinium tetrafluoroborate.

55. The process of claim 54, wherein aminoguanidinium tetrafluoroborate is prepared from aminoguanidinium bicarbonate according to scheme
Aminoguanidine-H⁺—HCO₃″+2HBF₄→Aminoguanidine-H₂²⁺—(BF₄⁻)₂+CO₂+H₂O (VI).

56. The process of claim 55, wherein aminoguanidinium tetrafluoroborate is prepared in situ in a third polar organic solvent.

57. The process of claim 56, wherein the third polar organic solvent is acetonitrile.

58. The process of claim 55, wherein water is removed from the reaction mixture by azeotropic distillation.

59. The process of claim 55, wherein aminoguanidinium tetrafluoroborate is not isolated before undergoing the subsequent reaction step.

60. A method of purifying a compound of formula 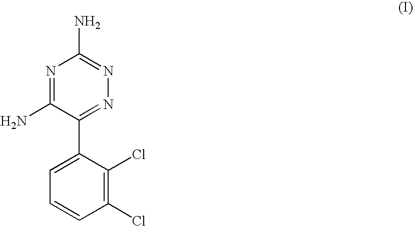
or a salt thereof, obtainable according to the process of claim 1, wherein said compound is crystallized from a mixture of isopropanol and water.

61. A method of claim 60, wherein said compound is crystallized from a mixture of isopropanol and water having a volume ratio of isopropanol:water of 3:1 to 2:1.

62. A method of claim 60, wherein said compound is obtained in an essentially anhydrous form.

63. A method of claim 60, wherein said compound is obtained in an essentially anhydrous form having a water content of less than 0.1% (w/w).

64. A salt of the stoichiometric composition L-X, wherein L is the singly protonated cation of compound 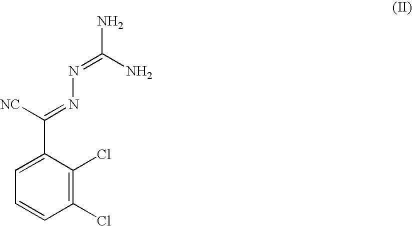
and wherein X is a singly negatively charged anion of an acid selected from the group consisting of sulfuric acid, phosphoric acid, polyphosphoric acids, metaphosphoric acids, tetrafluoroboric acid, tetrachloroboric acid, tetraalkylboric acids, tetraarylboric acids, and tetra(alkylaryl)boric acids.

65. A salt of claim 64, wherein X is a tetrafluoroborate or a tetraphenylborate ion.

66. A salt of claim 64, wherein X is a tetrafluoroborate ion.

67. A salt of the stoichiometric composition L₂-X, wherein L is the singly protonated cation of compound 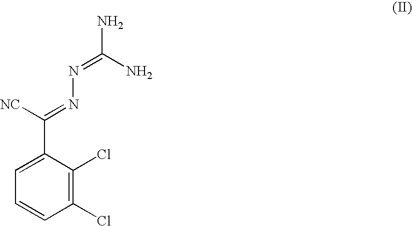
and wherein X is a doubly negatively charged anion of an acid selected from the group consisting of sulfuric acid, phosphoric acid, polyphosphoric acids, and metaphosphoric acids.

68. A salt of formula

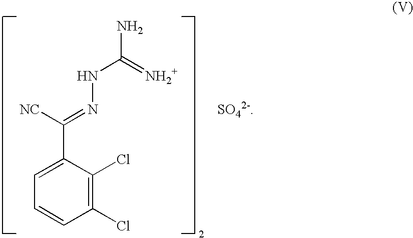

69. The process of claim 3, wherein in step (a) an amount of at least 0.5 equivalents of said dehydrating agent per equivalent of aminoguanidinium bicarbonate is added.

70. The process of claim 3, wherein in step (a) an amount of 1 to 1.5 equivalents of said dehydrating agent per equivalent of aminoguanidinium bicarbonate is added.

71. The process of claim 3, wherein the first and/or second polar solvent or solvent mixture is a polar aprotic organic solvent.

72. The process of claim 3, wherein the first and/or second polar solvent or solvent mixture is a water-miscible polar aprotic organic solvent.

73. The process of claim 3, wherein the first and/or second polar solvent or solvent mixture is selected from the group consisting of sulfolane, N-methylpyrrolidone, dimethylacetamide, dimethylformamide, tetrahydrofuran, dioxane, sulfur dioxide, dimethyl sulfoxide, and acetonitrile.

74. The process of claim 3, wherein the first polar solvent or solvent mixture also comprises an organic sulfonic acid or a mixture of organic sulfonic acids.

75. The process of claim 3, wherein the first and/or second polar solvent or solvent mixture comprises at least 3 equivalents of an organic sulfonic acid or a mixture of organic sulfonic acids per equivalent of aminoguanidine starting material.

76. The process of claim 3, wherein the first and/or second polar solvent or solvent mixture comprises at least 7 equivalents of an organic sulfonic acid or a mixture of organic sulfonic acids per equivalent of aminoguanidine starting material.

77. The process of claim 3, wherein the first and/or second polar solvent or solvent mixture comprises at least 9 equivalents of an organic sulfonic acid or a mixture of organic sulfonic acids per equivalent of aminoguanidine starting material.

78. The process of claim 3, wherein the second polar solvent is an organic sulfonic acid.

79. The process of claim 3, wherein both the first and the second polar solvents are an organic sulfonic acid.

80. The process of claim 3, wherein both the first and the second polar solvents are the same organic sulfonic acid.

81. A method of purifying a compound of formula 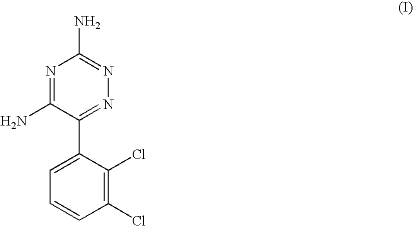
or a salt thereof, obtainable according to the process of claim 43, wherein said compound is crystallized from a mixture of isopropanol and water.
